(19) United States
(12) Patent Application Publication
(10) Pub. No.: US 2006/0094682 A1
(43) Pub. Date: May 4, 2006

(54) Kinase inhibitors for the treatment of diabetes and obesity

(75) Inventors: John K. Westwick, San Ramon, CA (US); Jane Lamerdin, Livermore, CA (US); Stephen Owens, Martinez, CA (US); Marnie L. MacDonald, Pleasanton, CA (US)

Correspondence Address: Isaac A. Angres, Suite 301, 2001 Jefferson Davis Highway, Arlington, VA, 22202, US

(73) Assignee: Odyssey Thera, Inc., San Ramon, CA (US)

(21) Appl. No.: 11/260,164

(22) Filed: Oct. 28, 2005

Related U.S. Application Data
(60) Provisional application No. 60/622,801, filed on Oct. 29, 2004.

(51) Int. Cl. A61K 48/00; A61K 38/54; A61K 39/395

(52) U.S. Cl. 514/44; 514/2; 424/146.1

(57) ABSTRACT

The present invention discloses a method of treating an individual or animal with diabetes and/or obesity. The method comprises administering to the individual or animal a therapeutically effective amount of a protein tyrosine kinase inhibitor. Preferably, the preventative and therapeutic methods of the present invention involve administering—to a mammal in need thereof—a therapeutically effective amount of an inhibitor of a c-Src-family protein tyrosine kinase. The invention pertains to pharmaceutical compositions containing an inhibitor of a c-Src-family protein tyrosine kinase or an analog or metabolite thereof, or an inhibitor of another protein tyrosine kinase, and a pharmaceutically acceptable carrier. Purines and pyrimidines and other molecules useful in the treatment of diabetes and obesity are provided herein, in particular, pyrazolopyrimidines, cyanoquinolines, phenylaminopyrimidines, anilinoquinazolines and related compounds. The invention also provides cellular targets and assay compositions useful for the identification of additional novel therapeutic agents for the treatment of these disorders.

This application claims the priority benefit under 35 U.S.C. section 119 of U.S. Provisional Patent Application No. 60/611,715 entitled “Kinase Inhibitors For The Treatment Of Diabetes And Obesity”, filed Oct. 29, 2004, which is in its entirety herein incorporated by reference.

### BACKGROUND OF THE INVENTION

Estimated to affect around a quarter of the adult population of the US, or some 50 million people, metabolic syndrome constitutes a major health problem. Metabolic syndrome, which is sometimes called insulin resistance syndrome, is characterized by a cluster of conditions which can include central obesity; high triglyceride and low HDL levels; raised blood pressure; insulin resistance or glucose intolerance; a pro-thrombic state; and a pro-inflammatory state. The presence of some or all of these risk factors predisposes individuals to an increased risk of cardiovascular diseases such as coronary heart disease, peripheral vascular disease and stroke, as well as Type 2 diabetes. Estimates suggest that more than half of all Type 2 diabetics have the characteristics of metabolic syndrome.

The currently-marketed treatments for type 2 diabetes are oral medications in the thiazolidinedione (TZD) class of compounds which are more commonly termed glitazones. They are marketed by SmithKline Beecham and Eli Lilly, respectively, under the names Avandia and Actos. TZDs increase insulin responsiveness in human and animal models of Type 2 diabetes. TZDs exert most, if not all, of their metabolic effects through the specific binding and activation of peroxisome proliferator-activated receptors (PPARs). PPARs belong to the nuclear receptor superfamily of ligand-activated transcription factors. The PPAR family comprises the closely related PPAR-[alpha], -[gamma], and -[beta]. PPARs transduce the effects of their ligands into transcriptional responses, and thereby function as important regulators in lipid and glucose metabolism, adipocyte differentiation, inflammatory response and energy homeostasis.

When activated by their cognate ligands, PPARs form a heterodimer with another steroid receptor, retinoid receptor X (RXR). This protein complex then induces the expression of metabolism-related genes through a direct interaction at specific DNA response elements or by binding to other transcription factors. Through these interactions, the PPAR proteins regulate the cellular response to both excess and shortage of dietary factors, an essential process for maintaining homeostasis.

The isoforms of PPAR each play a separate role in systemic metabolism. The tissue distribution of each is illustrative of this diversity of function: PPARα is found in the liver, heart, kidney, as well as other tissues where metabolism is elevated. PPAR-[alpha] is the molecular target for the fibrates, drugs that are widely prescribed for the reduction of elevated triglyceride levels. At the molecular level, fibrates regulate the transcription of a large number of genes that affect lipoprotein and fatty acid metabolism. PPARγ, on the other hand, is present predominantly in adipose tissue and tissues related to the immune system. PPAR-[Gamma] is the molecular target for the TZDs, including Rezulin (troglitazone) which was originally developed for the treatment of Type 2 diabetes; Avandia (rosiglitazone) and Actos (pioglitazone).PPARβ is more ubiquitously expressed than both α and γ. Its function is largely unknown. PPARα and γ have been identified as central regulators of critical biological processes. Disruption of PPAR function, for instance by mutation, such as the L162V mutation in PPARα and the P12A mutation in PPARγ, has been implicated in the development of such devastating human pathologies as cardiovascular disease, diabetes, and cancer. Diabetic patients suffer not only from the effects of hyperglycemia, but also from imbalances in linked metabolic pathways resulting in hypertriglyceridemia and hypercholesterolemia. Generally several drugs must be taken concurrently to fully manage the metabolic syndrome. Currently available treatment regimens are primarily directed at either lowering glucose with a glitazone or at lowering cholesterol, such as with a statin (Lipitor, Crestor or Zocor). Newer pan-PPAR-agonists, such as the novel small molecule PLX204 (Plexxikon, Inc.) modulate the function of three related targets, PPAR-[Alpha], -[Delta] and -[Gamma], with the goal of lowering glucose, triglycerides and free fatty acids, and increasing high-density lipoprotein (HDL).

Although PPAR-[Gamma] agonists have proven efficacy for reducing plasma glucose levels in patients with type 2 diabetes mellitus, they are not safe for all patients. Troglitazone, the first compound approved by the US Food and Drug Administration, was withdrawn from the market after the report of several dozen deaths or cases of severe hepatic failure requiring liver transplantation. It remains unclear whether or not hepatotoxicity is a class effect or is related to unique properties of troglitazone. All the TZDs have been linked to fluid retention, which can exacerbate or contribute to congestive heart failure. Past clinical studies have shown an increased incidence of heart failure and other cardiovascular adverse events in patients on Avandia or Actos plus insulin, compared with insulin alone.

Although these receptors constitute well-established therapeutic targets, they are “orphan” receptors in that their native ligand(s) are still unknown. However, their mechanism of activation by small-molecule ligands has been extensively characterized. The carboxy-terminal portion of the PPARs contains a ligand-binding domain which serves as a molecular switch that recruits co-activator proteins and activates the transcription of target genes when flipped into the active conformation by ligand binding. A growing number of coactivators and corepressors is being identified and characterized, suggesting precise combinatorial control of receptor function. Members of a family of 160-kDa proteins, referred to as the steroid receptor coactivator (SRC) family interact with PPARs in a ligand-dependent manner. The SRC family includes the proteins SRC1/NCOA1, TIF2/GRIP1, and pCIP/A1B1/ACTR/RAC/TRAM-1. As more than 30 additional putative cofactors have been identified, including proteins with protease activity and an RNA that appears to function as a co-activator, it is likely that different protein complexes can act either sequentially, combinatorially, or in parallel, particularly in light of the evidence of rapid turnover of DNA-receptor interactions.

The signal transduction pathway(s) controlling the activation of endogenous PPARs is also largely uncharacterized. A few clues have come from studies of the epidermal growth factor (EGF)-dependent pathway. The EGF receptor (EGFR) kinase is “transactivated” by a number of stimuli (G-protein receptor activation, ultraviolet radiation, peroxide, and other cell signals). What differentiates this from a typical signal—such as that of EGF itself—is that it is either ligand-independent or involves proteolytic release of EGF-like ligands from the cell surface. Recent studies demonstrated that glitazones induced EGFR transactivation in rat liver epithelial cells. In addition these compounds caused the rapid activation of the ERK and p38 MAPK's by both EGFR-dependent and -independent mechanisms. These studies suggested that PPAR agonists elicited “non-genomic” events that could influence cellular responses to these compounds.

Elucidation of the detailed mechanisms of action of PPARs, their regulation in human cells, and the pathways controlling their activity, will reveal entirely new biochemical mechanisms that can be targeted for therapeutic intervention. Importantly, the identification of new targets should enable the discovery of new classes of therapeutic agents with improved safety and efficacy in man.

### SUMMARY OF THE INVENTION

This invention relates to a method of treating or preventing disease with a Src- or Src-family inhibitor, which method comprises administering to a patient in need of such a treatment a therapeutically effective amount of a compound or a pharmaceutical composition thereof. Accordingly, the invention features a method for treating a patient having diabetes or obesity or a related condition or a complication thereof, other neoplasm, by administering to the patient an inhibitor of a protein tyrosine kinase in an amount sufficient to improve insulin sensitivity or to lower blood glucose or to assist in weight loss, whether administered alone or in combination with another pharmaceutical agent or in combination with with diet and exercise. The pharmaceutical compositions provided herein may be administered in conjunction with insulin and with lipid-lowering, cholesterol-lowering and/or anti-hypertensive agents. The conditions that may be ameliorated according to any of the methods of the invention, described below, include diabetes; obesity; hypertriglyceridemia; high blood pressure; insulin resistance or glucose intolerance; diabetic retinopathy; diabetic neuropathy; a pro-thrombic state; and a pro-inflammatory state. In addition the method of treatment includes the prevention, or delay in onset, of these conditions in individuals predisposed to these conditions. The invention also provides numerous chemical compounds that are known protein tyrosine kinase inhibitors, and specifically c-Src inhibitors, and core structures and scaffolds related thereto, that may be used in conjunction with the development of therapeutic agents for these conditions. The invention also provides small interfering RNAs, antisense and RNAi compositions for the treatment and prevention of diabetes and obesity and related complications.

The invention also features targets for drug discovery for diabetes and obesity; and methods for identifying additional therapeutic agents for the treatment of diabetes and obesity, specifically, cell-based assays that can be used in high-throughput and high-content screening to identify compounds that are themselves ligands of the PPARs or that act as surrogates, by acting upon targets upstream of the PPARs in cellular pathways.

### BRIEF DESCRIPTION OF THE DRAWINGS

FIG. 1. Construction of an assay for the detection of PPAR-[Gamma] activation. Rosiglitazone increases PPARgamma:SRC1 complexes in human cells as assessed by a fluorescence protein-fragment complementation assay (PCA) in human cells.

FIG. 2. Effects of individually silencing known genes on PPAR-[Gamma]:SRC-1 complexes in the presence of 15 micromolar rosiglitazone. Over 100 individual genes were silenced with siRNA Smart Pools (Dharmacon, Inc.) by co-transfecting each siRNA pool along with the PCA DNAs encoding the PPAR-[Gamma] and SRC-1 fragment fusions. Cells were stimulated with 15 □M rosiglitazone for 90 minutes prior to image analysis. Images were taken by automated microscopy and the fluorescence intensity of each image, representing the number of protein-protein complexes in the cells, was quantified by image analysis. Results are shown for each assay as % of the control siRNA. The greatest positive effect of silencing was observed for the siRNA targeting the non-receptor tyrosine kinase, c-Src. Silencing of PPAR-[Gamma] itself successfully knocked down PPAR-[Gamma], eliminating the complexes.

FIG. 3. Photomicrographs showing the effects of gene silencing in the absence and presence of rosiglitazone. In the presence of a control siRNA, rosiglitazone increases PPAR-[Gamma]:SRC-1 complexes in the cells (upper panels). An siRNA targeting PPAR-[Gamma] obliterates the PPAR-[Gamma]:SRC-1 complexes in the absence and presence of rosiglitazone (middle panels). An siRNA targeting the protein tyrosine kinase, c-Src, increases PPAR-[Gamma]:SRC-1 complexes even in the absence of rosiglitazone; and super-induces PPAR-[Gamma]:SRC-1 complexes in the presence of rosiglitazone (lower panels). Similar results were obtained in three independent experiments.

FIG. 4. A potent and selective chemical inhibitor of c-Src family kinases (PP2) mimics the effect of a c-Src siRNA on PPAR-[Gamma]. Kinase inhihibitors that are inactive against c-Src (PP3 and PD153035) have no effect on PPAR-[Gamma].

FIG. 5. Quantification of the effects of kinase inhibitors on PPAR-[Gamma]:SRC-1 in human cells. Methods were as described for FIG. 4. Data plotted for each drug treatment represent the mean (PPM) and standard error from 4 replicate wells in at least three independent experiments. Only the effect of PP2 was statistically significant (p<0.0001) relative to the DMSO control.

FIG. 6. Effects of kinase inhibitors and glitazones on the phosphorylation status of ERK. Left hand panel: Western blot of the phosphorylation status of p44/42 MAPK/ERK in HEK293 cells stimulated with EGF (Lane 1) or rosiglitazone (Lanes 2-6), in combination with PP2, PP3, PD 153035 or PD 98059. HEK293 cells were serum-starved overnight then pre-treated with DMSO, 10 micromolar PP2 or PP3, 1 micromolar PD 153035, or 20 micromolar PD 98059 for 1 hour prior to stimulation with rosiglitazone for 5 minutes. Cells stimulated with EGF (100 ng/ml for 5 minutes) served as a positive control. Right hand panels: Hep3B cells were serum starved overnight, then treated with PPAR-[Gamma] agonists rosiglitazone, troglitazone and ciglitazone (50 micromolar each) for the indicated times. The phosphorylation status of p44/42 MAPK/ERK was compared to that of unstimulated (basal) or vehicle-treated (DMSO) cell extracts.

FIG. 7 (A-G). Candidate compounds for the treatment of diabetes, obesity and other conditions associated with metabolic syndrome. Shown are representative compound names, structures, and mechanisms of action (where known) of c-Src kinase inhibitors that are candidate compounds for the treatment of metabolic syndrome disorders according to the present invention. The structure of PP2 (AG1879) is shown in FIG. 7C. Additional compounds useful in the present invention are provided in Table 3 and in the References, which are incorporated herein in their entirety.

### DETAILED DESCRIPTION OF THE INVENTION

The identification of new targets in the pathways controlling the PPAR family of transcription factors should enable the discovery of new classes of therapeutic agents with improved safety and efficacy in man. Gene silencing through RNA interference (RNAi) is finding immediate utility to the identification and validation of new therapeutic targets. Gene silencing also has long-term potential as a therapeutic strategy. RNAi strategies rely on the property of double-stranded RNA (dsRNA) to activate the endogenous cellular process of highly specific RNA degradation. If the silencing is effective, the protein encoded by the targeted RNAi will be knocked down; and the biochemical or phenotypic consequences of the knockdown can therefore be assessed. RNAi can thus be employed to link specific genes to their functional roles within the cellular signaling network and to identify proteins of potential relevance to a cellular process. If a gene has a positive or activating effect on a pathway, silencing that gene would be expected to block the positive modulation of the pathway. In contrast, if a gene has a negative or inhibitory effect on a downstream element in a pathway, silencing that gene would be expected to remove the block on the pathway.

Construction of an Assay to Measure PPAR Activation

We first constructed an assay for PPAR-[Gamma] in living cells. We used a protein-fragment complementation assay (PCA) designed to report on the complexes formed between PPAR-[Gamma] and its co-activator, SRC-1.

PCA involves fusion of full-length cellular proteins to fragments of a rationally-dissected reporter, such that interaction of the two proteins facilitates re-folding and activation of the reconstituted reporter. The PCA reporter utilized here is based on an intensely fluorescent variant of YFP, which enables detection of low levels of expressed protein as well as the real-time tracking of dynamic interactions and sub-cellular translocation. The PCA was designed such that a fluorescence signal forms when PPAR-[Gamma] interacts with SRC-1.

Reporter fragments for PCA were generated by oligonucleotide synthesis (Blue Heron Biotechnology, Bothell, Wash.). First, oligonucleotides coding for polypeptide fragments YFP[1]and YFP[2] (corresponding to amino acids 1-158 and 159-239 of YFP) were synthesized. Next, PCR mutagenesis was used to generate the mutant fragments IFP[1] and IFP[2]. The IFP[1] fragment corresponds to YFP[l]-(F46L, F64L, M153T) and the IFP[2] fragment corresponds to YFP[2]-(V163A, S175G). These mutations have been shown to increase the fluorescence intensity of the intact YFP protein (Nagai et al., 2002). The YFP[1], YFP[2], IFP[1] and IFP[2] fragments were amplified by PCR to incorporate restriction sites and a linker sequence, described below, in configurations that would allow fusion of a gene of interest to either the 5′- or 3′-end of each reporter fragment. The reporter-linker fragment cassettes were subcloned into a mammalian expression vector (pcDNA3.1 Z, Invitrogen) that had been modified to incorporate the replication origin (oriP) of the Epstein Barr virus (EBV). The oriP allows episomal replication of these modified vectors in cell lines expressing the EBNAI gene, such as HEK293E cells (293-EBNA, Invitrogen). Additionally, these vectors still retain the SV40 origin, allowing for episomal expression in cell lines expressing the SV40 large T antigen (e.g. HEK293T, Jurkat or COS). The integrity of the mutated reporter fragments and the new replication origin were confirmed by sequencing.

PCA fusion constructs were prepared for PPAR-[Gamma] and SRC-1 (Table 1), which are known to interact as components of the transcription complex. The full coding sequence for each gene was amplified by PCR from a sequence-verified full-length cDNA. Resulting PCR products were column purified (Centricon), digested with appropriate restriction enzymes to allow directional cloning, and fused in-frame to either the 5′ or 3′-end of YFP[l], YFP[2], IFP[1] or IFP[2] through a linker encoding a flexible 10 amino acid peptide (Gly.Gly.Gly.Gly.Ser)2. The flexible linker ensures that the orientation/arrangement of the fusions is optimal to bring the reporter fragments into close proximity (Pelletier et al., 1998). Recombinants in the host strains DH5-alpha (Invitrogen, Carlsbad, Calif.) or XL1 Blue MR (Stratagene, La Jolla, Calif.) were screened by colony PCR, and clones containing inserts of the correct size were subjected to end sequencing to confirm the presence of the gene of interest and in-frame fusion to the appropriate reporter fragment. A subset of fusion constructs were selected for full-insert sequencing by primer walking. DNAs were isolated using Qiagen MaxiPrep kits (Qiagen, Chatsworth, Calif.). PCR was used to assess the integrity of each fusion construct, by combining the appropriate gene-specific primer with a reporter-specific primer to confirm that the correct gene-fusion was present and of the correct size with no internal deletions.

TABLE 1
Protein-fragment complementation assay used in the invention

| | | | | Reporter 1 | | Reporter 2 |
|---|---|---|---|---|---|---|
| Assay | | Stimulus, | Gene 1 | Fusion | Gene 2 | Fusion |
| # | PCA Pair | conc (time) | Accession | Orientation | Accession | Orientation |
| 23 | PPAR- | Rosiglitazone, | NM_138712 | C | U40396 (nt | N |
| | [Gamma]:SRC-1 | 15 micromolar | | | 624 . . . 1256) | |

HEK293 cells were maintained in MEM alpha medium (Invitrogen) supplemented with 10% FBS (Gemini Bio-Products), 1% penicillin, and 1% streptomycin, and grown in a 37° C. incubator equilibrated to 5% CO₂. Approximately 24 hours prior to transfections cells were seeded into 96 well ploy-D-Lysine coated plates (Greiner) using a Multidrop 384 peristaltic pump system (Thermo Electron Corp., Waltham, Mass.) at a density of 7,500 cells per well. Up to 100 ng of the complementary fragment-fusion vectors were co-transfected using Fugene 6 (Roche) according to the manufacturer's protocol. Following 48 hours of expression, cells were tested for the presence of a fluorescence signal.

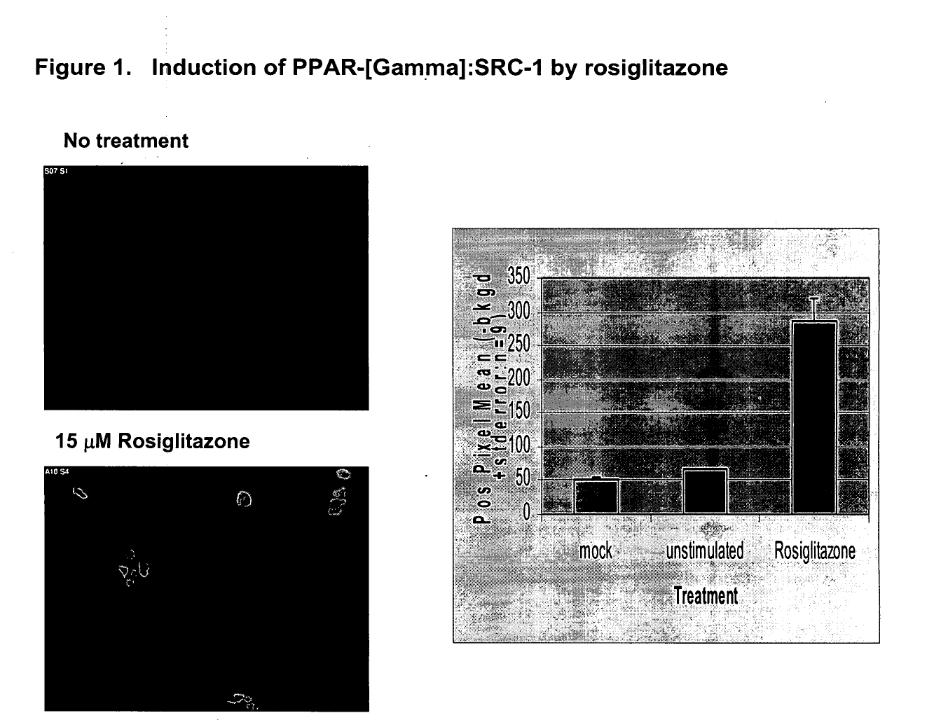

As shown in FIG. 1, in the absence of treatment (mock transfection or unstimulated) there was a low level of fluorescence in a limited number of cells. Stimulation with 15 micromolar rosiglitazone for 90 minutes increased the fluorescence intensity of the assay 6-fold, indicating an increase in the formation of complexes between PPAR-[Gamma] and its coactivator, SRC-1, as would be expected. These results demonstrate that the chosen assay faithfully reports the activity of PPAR in live cells response to a known ligand.

Identification of Targets Linked to PPAR Activation

We next sought to identify genes which, when silenced, would mimic the effect of rosiglitazone on PPAR-[Gamma]. Such genes would therefore represent negative modulators of PPAR-[Gamma] which could serve as surrogate targets for drug discovery. If such targets were drug-able, small molecule inhibitors could be found that would indirectly activate PPAR-[Gamma] as assessed by an increase in the PPAR-[Gamma]:SRC-1 complex (referred to hereafter as PPAR:SRC for brevity). Such molecules would therefore be surrogates for rosiglitazone and other TZD and non-TZD activators of PPARs.

We systematically silenced a large and diverse set of therapeutically relevant targets within cell signaling networks, and assessed the effects on the PPAR:SRC complex in intact human (HEK293) cells. A panel comprising 107 targeted siRNA pools was designed to target components of key signaling pathways and processes in the cell (see Table 2), including the specific PI3K/Akt-, RAS/MAPK- and NF[Kappa]B-mediated pathways; and pathways underlying DNA damage response, cell cycle, apoptotic regulators and nuclear hormone receptor signaling. Individually, the genes that were silenced code for receptors, adaptors, protein kinases and phosphatases, heat shock proteins, histone deacetylases, ubiquitin ligases, cell cycle and cytoskeletal proteins.

TABLE 2
Panel of 107 siRNAs used for gene silencing.

| siRNA | | | | | Dharmacon |
|---|---|---|---|---|---|
| No. | siRNA Name | Protein Target | Pathway/Classification | Gene Accession | Product Number |
| 1 | PTEN | PTEN | PI3K/AKT | NM_000314 | M-003023-00-05 |
| 2 | PIK3CA | p110a PI3K | PI3K/AKT | NM_006218 | M-003018-00-05 |
| 3 | PIK3R1 | p85a PI3K | PI3K/AKT | NM_181523 | M-003020-00-05 |
| 4 | PDPK1 | Pdk1 | PI3K/AKT | NM_002613 | M-003558-00-05 |
| 5 | AKT1 | Akt1 | PI3K/AKT | NM_005163 | M-003000-00-05 |
| 6 | AKT2 | Akt2 | PI3K/AKT | NM_001626 | M-003001-00-05 |
| 7 | GSK3B | Gsk3b | PI3K/AKT | NM_002093 | M-003010-00-05 |
| 8 | RPS6KB1 | p70S6K | PI3K/AKT | NM_003161 | M-003616-00-05 |
| 9 | FRAP1 | FRAP/TOR | PI3K/AKT | NM_004958 | M-003008-01-05 |
| 10 | FKBP | FK506-BP (12 kD) | PI3K/AKT | NM_054014 | M-005183-00-05 |
| 11 | HSPCA | Hsp90a | Hsp90/co-chaperones | NM_005348 | M-005186-00-05 |
| 12 | HSPCB | Hsp90b | Hsp90/co-chaperones | NM_007355 | M-005187-00-05 |
| 13 | CDC37 | Cdc37 | Hsp90/co-chaperones | NM_007065 | M-003231-00-05 |
| 14 | TEBP | P23 | Hsp90/co-chaperones | NM_006601 | M-005192-00-05 |
| 15 | cIAP1 | cIAP1 | Apoptosis | NM_001166 | M-004390-00-05 |
| 16 | cIAP2 | cIAP2 | Apoptosis | NM_001165 | M-004099-00-05 |
| 17 | Smac/Diablo | Smac/Diablo | Apoptosis | NM_019887 | M-004447-00-05 |
| 18 | BCL2 | BCL2 | Apoptosis | NM_000633 | M-003307-00-05 |
| 19 | BCL-xL | BCL-xL | Apoptosis | NM_138578 | M-003458-00-05 |
| 20 | TNFR1 | TNF-R | NFkB signaling | NM_001065 | M-005197-00-05 |
| 21 | RIP2 | RIP2 | NFkB signaling | NM_003821 | M-005370-00-05 |
| 22 | RIP4 | RIP4 | NFkB signaling | NM_020639 | M-005308-00-05 |
| 23 | TRADD | TRADD | NFkB signaling | NM_003789 | M-004452-00-05 |
| 24 | FADD | FADD | NFkB signaling | NM_003824 | M-003800-00-05 |
| 25 | TRAF2 | TRAF2 | NFkB signaling | NM_021138 | M-005198-00-05 |
| 26 | TRAF6 | TRAF6 | NFkB signaling | NM_004620 | M-004712-00-05 |
| 27 | IKBKA | IKKa | NFkB signaling | NM_001278 | M-003473-00-05 |
| 28 | IKBKB | IKKb | NFkB signaling | XM_032491 | M-004120-00-05 |
| 29 | IKBKE | IKKe | NFkB signaling | NM_014002 | M-003723-00-05 |
| 30 | NFKBIA | IkBa | NFkB signaling | NM_020529 | M-004765-00-05 |
| 31 | NFKB1B | IkBb | NFkB signaling | NM_002503 | M-004764-00-05 |
| 32 | RELA/p65 | NFkB-p65 | NFkB signaling | NM_021975 | M-003533-00-05 |
| 33 | NFKB-p50 | NFkB-p50 | NFkB signaling | NM_003998 | M-003520-00-05 |
| 34 | CREBBP | CBP | NFkB signaling | NM_004380 | M-003477-00-05 |
| 35 | HDAC1 | HDAC1 | Nuclear Hormone Receptor | NM_004964 | M-003494-00-05 |
| 36 | HDAC2 | HDAC2 | Nuclear Hormone Receptor | NM_001527 | M-003495-00-05 |
| 37 | SRC-1 | SRC-1 | Nuclear Hormone Receptor | U90661.1 | M-005196-00-05 |
| 38 | ESR1 | ERa | Nuclear Hormone Receptor | NM_000125 | M-003489-00-05 |
| 39 | PPARG | PPARg | Nuclear Hormone Receptor | NM_138712 | M-003436-00-05 |
| 40 | RXRA | RXRa | Nuclear Hormone Receptor | NM_002957 | M-003443-00-05 |
| 41 | SKP2 | Skp2 | Cell cycle/damage response | NM_005983 | M-003541-00-05 |
| 42 | b-TRCP | □TRCP | Cell cycle/damage response | NM_033637 | M-003463-00-05 |
| 43 | MDM2 | Hdm2 | Cell cycle/damage response | NM_002392 | M-003279-00-05 |
| 44 | TP53 | p53 | Cell cycle/damage response | NM_000546; | M-003329-00-05 |
| | | | | M14695 | |
| 45 | ATM | ATM | Cell cycle/damage response | NM_000051 | M-003201-00-05 |
| 46 | ATR | ATR | Cell cycle/damage response | NM_001184 | M-003202-01-05 |
| 47 | ABL1 | c-ABL | Cell cycle/damage response | NM_007313 | M-003100-01-05 |
| 48 | BRCA1 | Brca1 | Cell cycle/damage response | NM_007295 | M-003461-00-05 |
| 49 | CHEK1 | Chk1 | Cell cycle/damage response | NM_001274 | M-003255-01-05 |
| 50 | CHEK2 | Chk2 | Cell cycle/damage response | NM_007194 | M-003256-00-05 |
| 51 | CDC25A | Cdc25A | Cell cycle/damage response | NM_001789 | M-003226-00-05 |
| 52 | CDC25C | Cdc25C | Cell cycle/damage response | NM_001790 | M-003228-00-05 |
| 53 | PLK | Plk | Cell cycle/damage response | NM_005030 | M-003290-00-05 |
| 54 | CDK4 | Cdk4 | Cell cycle/damage response | NM_000075 | M-003238-00-05 |
| 55 | RB1 | Rb | Cell cycle/damage response | NM_000321 | M-003296-00-05 |
| 56 | CDKN1A | Cip/p21 | Cell cycle/damage response | NM_078467; | M-003471-00-05 |
| | | | | NM_000389 | |
| 57 | CDKN1B | Kip/p27 | Cell cycle/damage response | NM_004064 | M-003472-00-05 |
| 58 | CDKN2A | INK4/p16 | Cell cycle/damage response | NM_000077 | M-005191-00-05 |
| 59 | 14-3-3s | 14-3-3s | Cell cycle/damage response | NM_006142 | M-005180-00-05 |
| 60 | STAT1 | Stat1 | Ras/MAPK | NM_007315 | M-003543-00-05 |
| 61 | JAK1 | Jak1 | Ras/MAPK | NM_002227 | M-003145-01-05 |
| 62 | EGFR | EGFR | Ras/MAPK | NM_005228 | M-003114-01-05 |
| 63 | SRC | c-Src | Ras/MAPK | NM_005417 | M-003175-01-05 |
| 64 | GRB2 | Grb2 | Ras/MAPK | NM_002086 | M-004112-00-05 |
| 65 | SOS1 | Sos1 | Ras/MAPK | NM_005633 | M-005194-00-05 |
| 66 | SOS2 | Sos2 | Ras/MAPK | XM_043720 | M-005195-00-05 |
| 67 | PLCG1 | PLC-g | Ras/MAPK | NM_002660 | M-003559-00-05 |
| 68 | RalGDS | RalGDS | Ras/MAPK | NM_006266 | M-005193-00-05 |
| 69 | RAS | H-Ras | Ras/MAPK | NM_005343 | M-004142-00-05 |
| 70 | KRAS2 | K-Ras | Ras/MAPK | NM_004985 | M-005069-00-05 |
| 71 | RAF1 | c-Raf | Ras/MAPK | NM_002880 | M-003601-00-05 |
| 72 | B-Raf | B-Raf | Ras/MAPK | NM_004333 | M-003460-00-05 |
| 73 | MEK1 | Mek1 | Ras/MAPK | NM_002755 | M-003571-00-05 |
| 74 | MEK2 | Mek2 | Ras/MAPK | NM_030662 | M-003573-00-05 |
| 75 | ERK2 | Erk2 | Ras/MAPK | M84489 | M-003555-02-05 |
| 76 | ERK1 | Erk1 | Ras/MAPK | AK091009 | M-003592-00-05 |
| 77 | ELK1 | Elk1 | Ras/MAPK | NM_005229 | M-003885-00-05 |
| 78 | VAV1 | Vav1 | Rho family | NM_005428 | M-003935-00-05 |
| 79 | CDC42 | Cdc42 | Rho family | NM_001791 | M-005057-00-05 |
| 80 | RAC1 | Rac1 | Rho family | NM_018890 | M-003560-00-05 |
| 81 | PAK1 | Pak1 | Rho family | NM_002576 | M-003521-00-05 |
| 82 | PAK2 | Pak2 | Rho family | NM_002577 | M-003597-00-05 |
| 83 | PAK3 | Pak3 | Rho family | AF068864 | M-003614-00-05 |
| 84 | PAK4 | Pak4 | Rho family | NM_005884 | M-003615-00-05 |
| 85 | RhoA | RhoA | Rho family | NM_001664 | M-004549-00-05 |
| 86 | ROCK1 | p160-ROCK | Rho family | NM_005406 | M-003536-00-05 |
| 87 | MAP3K1 | MEKK1 | JNK/SAPK signaling | XM_042066 | M-003575-00-05 |
| 88 | MAP2K7 | MKK7/JNKK2 | JNK/SAPK signaling | NM_005043 | M-004016-00-05 |
| 89 | ASK1 | MEKK5 | JNK/SAPK signaling | E14699 | M-004539-00-05 |
| 90 | MAP2K4 | MKK4/JNKK1 | JNK/SAPK signaling | NM_003010 | M-003574-00-05 |
| 91 | JNK2 | JNK2 | JNK/SAPK signaling | L31951 | M-003766-00-05 |
| 92 | JNK1 | JNK1 | JNK/SAPK signaling | L26318 | M-003765-00-05 |
| 93 | ITGa4 | ITGa4 | Ras/MAPK | L12002 | M-005189-00-05 |
| 94 | PTK2 | FAK | Ras/MAPK | NM_005607 | M-003164-01-05 |
| 95 | CTNNB1 | □catenin | Wnt pathway | NM_001904 | M-003482-00-05 |
| 96 | DVL1 | Dsh1 | Wnt pathway | U46461 | M-004068-00-05 |
| 97 | DVL2 | Dsh2 | Wnt pathway | NM_004422 | M-004069-00-05 |
| 98 | EDG4 | Edg-4/LPA2 | GPCR/G-protein | AF233092 | M-004602-00-05 |
| 99 | EDG7 | Edg-7/LP-A3 | GPCR/G-protein | NM_012152 | M-004895-00-05 |
| 100 | GNA13 | Gai-3 | GPCR/G-protein | NM_006496 | M-005184-00-05 |
| 101 | GLUT4 | GLUT4 | PKA/PKC signaling | NM_001042 | M-005185-00-05 |
| 102 | PPP2CB | PP2CB | Phosphatase | NM_004156 | M-003599-00-05 |
| 103 | PPP2CA | PP2CA | Phosphatase | NM_002715 | M-003598-00-05 |
| 104 | PKC | PKCa | PKA/PKC signaling | NM_002737 | M-003523-00-05 |
| 105 | PRKACG | PKA C-g | PKA/PKC signaling | NM_002732 | M-004651-00-05 |
| 106 | PRKACB | PKA C-b | PKA/PKC signaling | NM_002731 | M-004650-00-05 |
| 107 | AKAP | AKAP1/PRKA1 | PKA/PKC signaling | NM_003488 | M-005181-00-05 |

107 siRNA SMART pools designed to target the above genes and two ‘GC-match’ non-specific siRNAs (Dharmacon, Boulder, Colo.) were resuspended per the manufacturer's recommendations. PCA fusion-reporter constructs were produced as described above. Transfections were performed in HEK293 cells with 100 ng of nucleic acid per well (up to 50 ng of each fusion construct, and the appropriate siRNA SMART pool at 40 nM final concentration) with Lipofectamine 2000 (Invitrogen). For each screen, transfections were aliquotted in triplicate such that the assay containing the PPAR:SRC PCA spanned four 96-well plates. Each 96-well plate contained five internal controls: mock (no PCA), no siRNA, non-specific siRNA controls 1× and XI (47% and 36% GC content, respectively), and a PCA-specific control (to confirm degree of stimulation for assays treated with agonists). Optimal siRNA concentration was determined by evaluating the effects of siGFP (Dharmacon) and the non-specific siRNA controls on four different PCAs (data not shown).

Forty-eight hours after transfection, cells were fixed and stained with Hoechst prior to image acquisition on a Discovery-1 automated fluorescence imager (Molecular Devices, Inc.). Four non-overlapping populations (scans) of cells per well were obtained with the following filter sets: excitation 480/40 nm, emission 535/50 nm (YFP); excitation 360/40 nm, emission 465/30 nm (Hoechst). A constant exposure time for each wavelength was used to acquire all images for a given assay. Raw images in 16-bit grayscale TIFF format were analyzed using modules from the ImageJ API/library (http://rsb.info.nih.gov/ij/, NIH, MD). Based on training sets of images for each assay, three algorithms were evaluated to identify the one that best suited a specific assay. Images from the Hoechst and YFP channels were normalized using a rolling-ball algorithm [45] followed by thresholding in each channel to separate the foreground from the background. An iterative algorithm based on Particle Analyzer (ImageJ) was applied to the thresholded Hoechst image (THI) to generate a nuclear mask. The THI was used to define a nuclear mask (NM), and all positive pixels from the YI above a user-defined threshold that fell within the NM were sampled. The sum of the positive pixels was corrected for the threshold value, and normalized to the area of the THI, resulting in the ‘Nuc Sum’. The Nuc Sum for each sample represents the mean from at least twelve scans after application of a 2SD filter to exclude scans with fluorescent artifacts. Statistical significance of the effect of each siRNA was determined by performing single factor ANOVA on a minimum of three wells for each sample, using a p-value of ≦0.05 as significant. Significant effects (>40% change from control and p≦0.05) detected in the initial screen were repeated in triplicate in two additional transfections.

Results of Systematic Gene Silencing

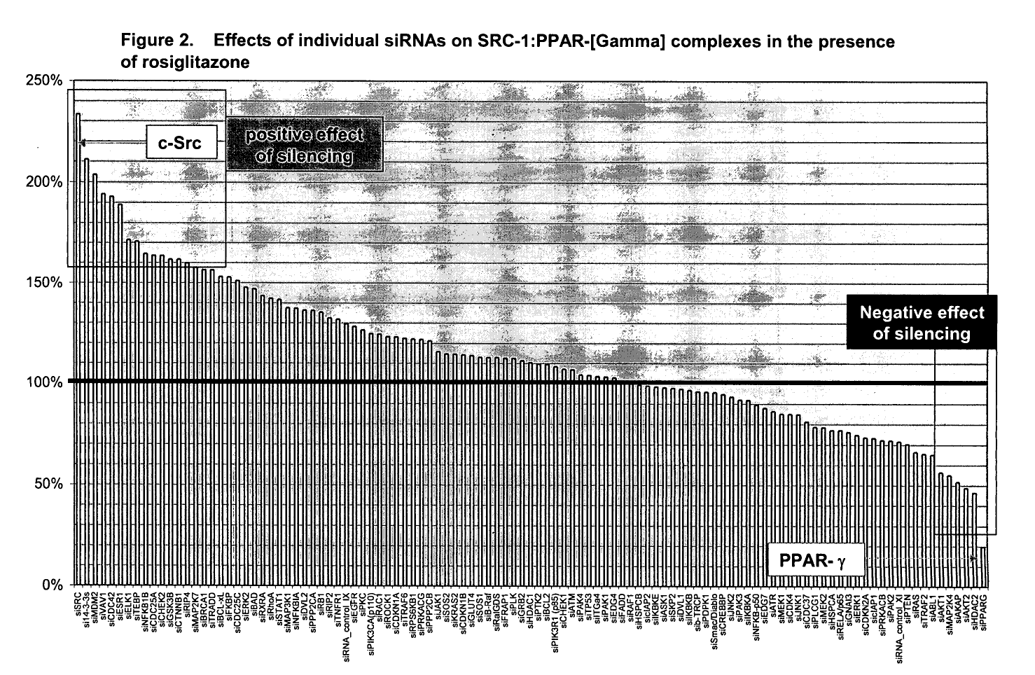

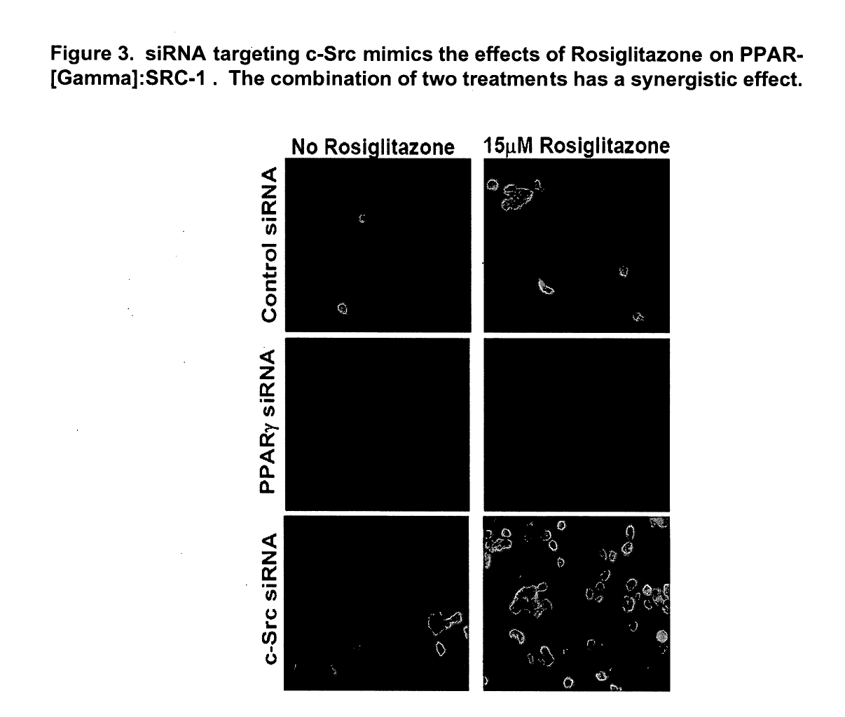

FIG. 2 shows the effects on PPAR-[Gamma]:SRC-1 of silencing individual genes, with the results ranked from left to right according to whether the gene silencing increased or decreased the number of PPAR:SRC complexes. Silencing of PPAR itself represented a control which, as expected, eliminated the signal from the PPAR:SRC PCA. As shown in FIG. 2, we observed the most dramatic induction of PPAR:SRC signaling complexes by silencing of the non-receptor tyrosine kinase c-src. (With respect to terminology, the proto-oncogene c-src is completely different from the nuclear receptor co-activator, SRC-1, even though the acronyms are similar). In the presence of rosiglitazone, siRNA-mediated knockdown of c-src resulted in a more than 8-fold increase in PPAR:SRC compared to control siRNA. Similar effects were obtained for the PPAR-[Gamma]:RXR-[Alpha] complex (not shown), indicating the effect was mediated through PPAR-[Gamma].

c-Src Link to PPAR-[Gamma] Involves a Novel Pathway

Our results suggest that c-Src plays a significant role in modulating the activity of PPAR and modulation of this effect does not occur via the EGFR/MAP kinase pathways, nor does it involve c-Src or EGFR/ERK activation by nuclear receptor agonists. These data are the first to directly demonstrate a novel pathway involving c-Src-mediated regulation of PPAR.

Known Roles of c-Src and Src Family Kinases

Since there is intense interest in activating PPAR as a strategy for treating metabolic and proliferative disorders, the identification of a completely novel link between c-Src and PPAR provides an additional drug-able target for therapeutic intervention. The Src kinase—and other nonreceptor tyrosine kinases—have never before been linked to PPAR activation; or to diabetes, obesity, or other conditions related to metabolic syndrome, nor has any other non-receptor protein tyrosine kinase. A brief background on c-Src is given here.

c-Src is a protein tyrosine kinase. Tyrosine kinases are enzymes that catalyze the transfer of the terminal phosphate of adenosine triphosphate to tyrosine residues in protein substrates. Tyrosine kinases are believed, by way of substrate phosphorylation, to play critical roles in signal transduction for a number of cell functions. Though the exact mechanisms of signal transduction is still unclear, tyrosine kinases have been shown to be important contributing factors in cell proliferation, carcinogenesis and cell differentiation.

Tyrosine kinases can be categorized as receptor type or non-receptor type. Receptor type tyrosine kinases have an extracellular, a transmembrane, and an intracellular portion, while non-receptor type tyrosine kinases are wholly intracellular. The receptor-type tyrosine kinases are comprised of a large number of transmembrane receptors with diverse biological activity. In fact, about twenty different subfamilies of receptor-type tyrosine kinases have been identified. One tyrosine kinase subfamily, designated the HER subfamily, is comprised of EGFR, HER2, HER3, and HER4. Ligands of this subfamily of receptors include epithileal growth factor, TGF-.alpha., amphiregulin, HB-EGF, betacellulin and heregulin. Another subfamily of these receptor-type tyrosine kinases is the insulin subfamily, which includes INS-R, IGF-R, and IR-R. The PDGF subfamily includes the PDGF-.alpha. and beta. receptors, CSFIR, c-kit and FLK-II. Then there is the FLK family which is comprised of the kinase insert domain receptor (KDR), fetal liver kinase-1 (FLK-1), fetal liver kinase-4 (FLK-4) and the fins-like tyrosine kinase-1 (flt-1). The PDGF and FLK families are usually considered together due to the similarities of the two groups. For a detailed discussion of the receptor-type tyrosine kinases, see Plowman et al., DN & P 7(6):334-339, 1994, which is hereby incorporated by reference.

The non-receptor type of tyrosine kinases is comprised of numerous subfamilies, including Src, Frk, Btk, Csk, Abl, Zap70, Fes/Fps, Fak, Jak, Ack, and LIMK. Each of these subfamilies is further sub-divided into varying receptors. The Src subfamily is one of the largest and includes Src, Yes, Fyn, Lyn, Lck, Blk, Hck, Fgr, and Yrk. The Fak family includes Pyk2. Tyrosine kinase-dependent diseases and conditions are generally thought to include angiogenesis, cancer, tumor growth, atherosclerosis, age-related macular degeneration, inflammatory diseases, and the like. For a more detailed discussion of the non-receptor type of tyrosine kinases, see Bolen Oncogene, 8:2025-2031 (1993), which is hereby incorporated by reference. Diabetes and obesity have never before been linked to tyrosine kinases.

The Src subfamily of enzymes has been linked to oncogenesis. Other Src-mediated conditions include hypercalcemia, osteoporosis, osteoarthritis, cancer, symptomatic treatment of bone metastasis, and Paget's disease. Src protein kinase and its implication in various diseases has been described [Soriano, Cell, 69, 551 (1992); Soriano et al., Cell, 64, 693 (1991); Takayanagi, J. Clin. Invest., 104, 137 (1999); Boschelli, Drugs of the Future 2000, 25(7), 717, (2000); Talamonti, J. Clin. Invest., 91, 53 (1993); Lutz, Biochem. Biophys. Res. 243, 503 (1998); Rosen, J. Biol. Chem., 261, 13754 (1986); Bolen, Proc. Natl. Acad. Sci. USA, 84, 2251 (1987); Masaki, Hepatology, 27, 1257 (1998); Biscardi, Adv. Cancer Res., 76, 61 (1999); Lynch, Leukemia, 7, 1416 (1993); Wiener, Clin. Cancer Res., 5, 2164 (1999); Staley, Cell Growth Diff., 8, 269 (1997)].

All Src-family kinases contain an N-terminal myristoylation site followed by a unique domain characteristic of each individual kinase, an SH3 domain that binds proline-rich sequences, an SH2 domain that binds phosphotyrosine-containing sequences, a linker region, a catalytic domain, and a C-terminal tail containing an inhibitory tyrosine. The activity of Src-family kinases is tightly regulated by phosphorylation. Two kinases, Csk and Ctk, can down-modulate the activity of Src-family kinases by phosphorylation of the inhibitory tyrosine. This C-terminal phosphotyrosine can then bind to the SH2 domain via an intramolecular interaction. In this closed state, the SH3 domain binds to the linker region, which then adopts a conformation that impinges upon the kinase domain and blocks catalytic activity. Dephosphorylation of the C-terminal phosphotyrosine by intracellular phosphatases such as CD45 and SHP-1 can partially activate Src-family kinases. In this open state, Src-family kinases can be fully activated by intermolecular autophosphorylation at a conserved tyrosine within the activation loop.

Small-Molecule Inhibitors of c-Src Mimic the Effect of Gene Silencing

Because of the novel link between c-Src and PPAR-[Gamma], we assessed whether the effects of silencing c-Src could be mimicked with a small-molecule inhibitor of the c-Src kinase. If so, such inhibitors would constitute alternative approaches to activating PPAR in human cells and therefore would constitute alternatives to thiazolidinediones for the treatment of similar disorders.

We used PP2 as a model compound for these studies. PP2 (4-amino-5-(4-chlorophenyl)-7-(t-butyl)pyrazolo[3,4-d]pyrimidine) is a potent, Src family-selective tyrosine kinase inhibitor (Hanke, J. H. et al. 1996: J Biol Chem 271, 695-701). It inhibits p56^{Ick} (IC₅₀=4 nM), p59^{fyn}T (IC₅₀=5 nM), and Hck (IC₅₀=5 nM). PP2 does not significantly affect the activity of EGFR kinase (IC₅₀=480 nM), JAK2 (IC₅₀>50 mM), or ZAP-70 (IC₅₀>100 mM). PP2 also inhibits the activation of focal adhesion kinase and its phosphorylation at Tyr⁵⁷⁷; and potently inhibits anti-CD3-stimulated tyrosine phosphorylation of human T cells (IC₅₀=600 nM) (Kami, R., et al. 2003. FEBS Lett. 537, 47. Salazar, E. P., and Rozengurt, E. 1999. J. Biol. Chem. 274, 28371). PP3, which is the compound 4-Amino-7-phenylpyrazol[3,4-d]pyrimidine, is a negative control for the Src family protein tyrosine kinase inhibitor PP2. Although PP3 is inactive against Src family kinases, it inhibits the activity of EGFR kinase (IC₅₀=2.7 mM (Traxler, P., et al. 1997. J. Med. Chem. 40, 3601)

PD153035 (AG 1517) is the compound 4-[(3-Bromophenyl)amino]-6,7-dimethoxyquinazoline, which is an extremely potent and specific inhibitor of the tyrosine kinase activity of the epidermal growth factor receptor (EGFR; IC₅₀=25 pM; Kᵢ=6 pM). PD153035 rapidly suppresses the autophosphorylation of EGFR at low nanomolar concentrations in fibroblasts or in human epidermoid carcinoma cells. It also selectively blocks EGF-mediated cellular processes including mitogenesis, early gene expression, and oncogenic transformation. (Bridges, A. J., et al. 1996. J. Med. Chem. 39, 267; Fry, D. W., et al. 1994, Science 265, 1093).

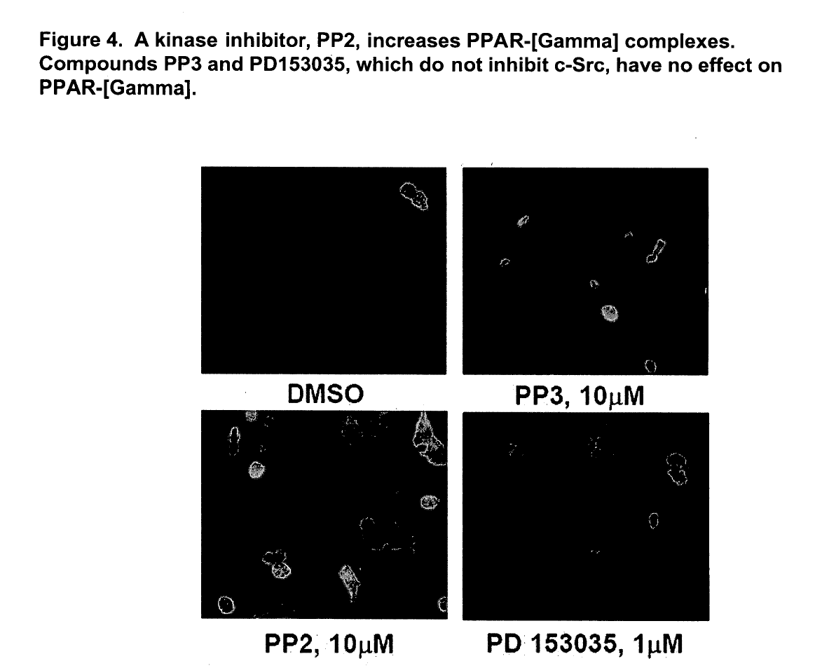

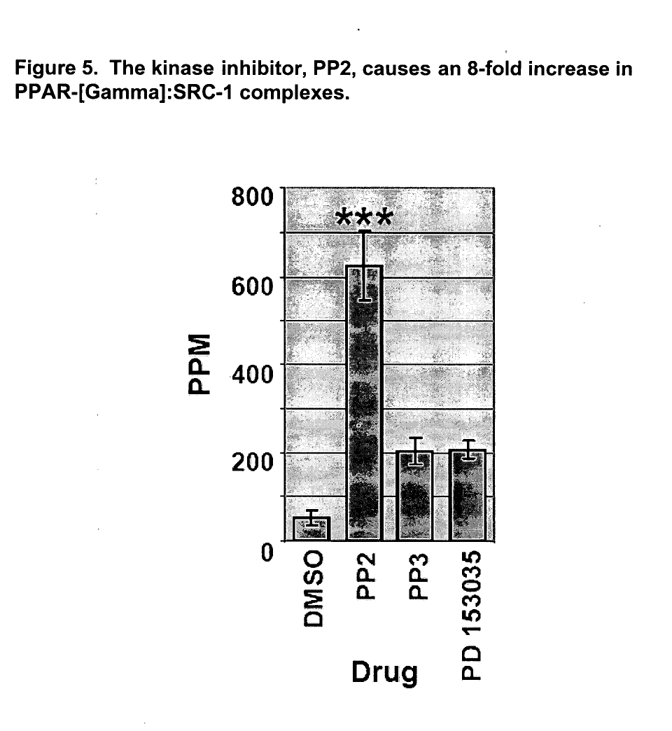

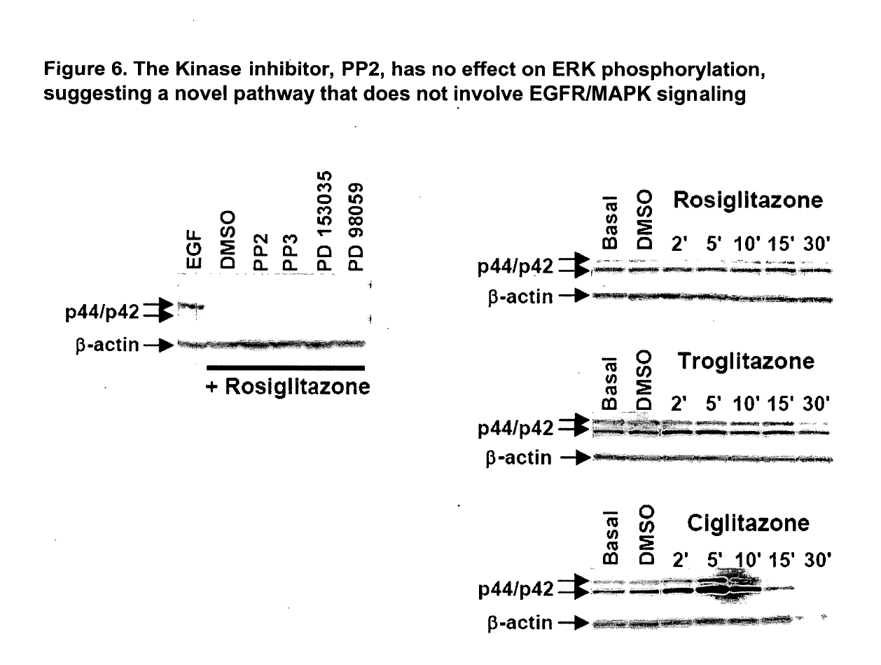

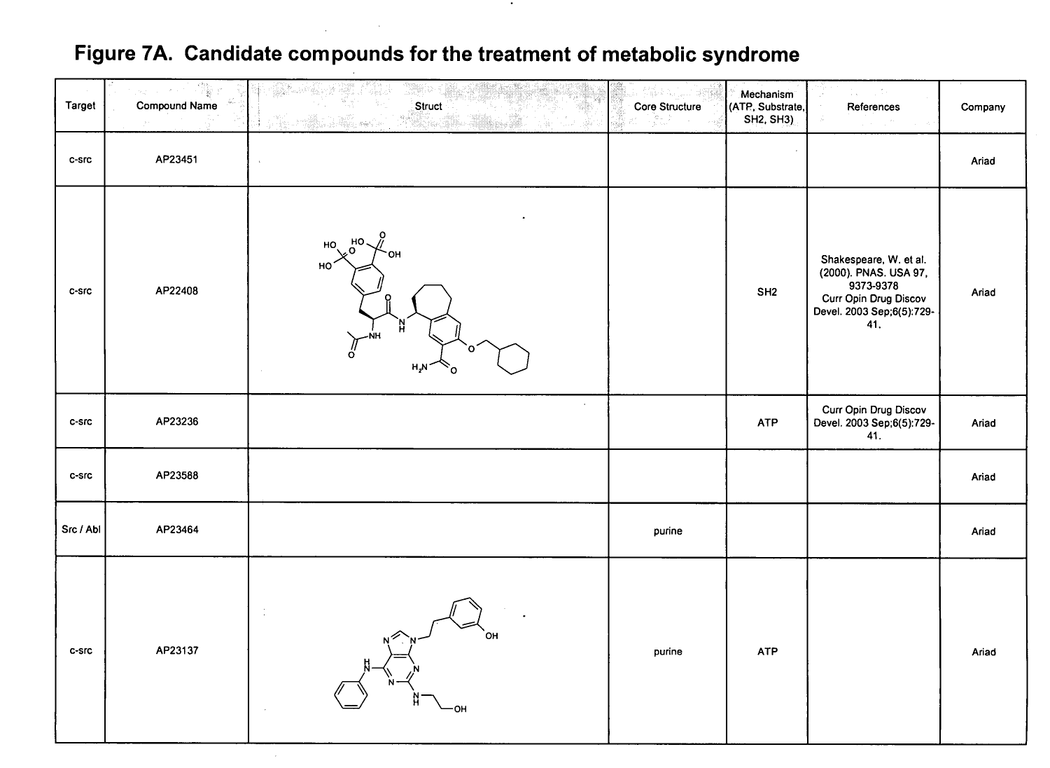

HEK293 cells transiently transfected with the PPAR-[Gamma]:SRC-1 PCA were serum-starved for 16 hours then treated with 10 micromolar PP2, 10 micromolar PP3, 1 micromolar PD 153035 or with vehicle for 6.5 hours prior to stimulation with rosiglitazone for 1.5 hours. Representative images for each drug are shown in FIG. 4. PP2 increased the PPAR:SRC complex 7-fold (p<0.0001). The structurally similar analog of PP2 which is inactive against c-Src (PP3) had no effect on PPAR-[Gamma] showing that the effects of PP2 and the c-Src siRNA were a direct result of c-Src inhibition. PD153035 also had no effect on PPAR:SRC suggesting a mechanism of action that is not mediated by EGF.

Candidate Compounds for the Treatment of Diabetes and Obesity and Related Conditions

Candidate compounds for the treatment of a disease are compounds that mimic the effects of known modulators of the disease process. In this case we have identified inhibitors of c-Src that mimic the effects of the thiazolidinediones on PPAR-[Gamma] in living human cells. Since TZDs have been proven to be effective in the treatment of metabolic syndrome, c-Src inhibition offers an alternate route to the treatment of the syndrome. In particular, we provide herein lead compounds that are Src-family-selective and have adequate pharmacokinetic and pharmacodynamic properties.

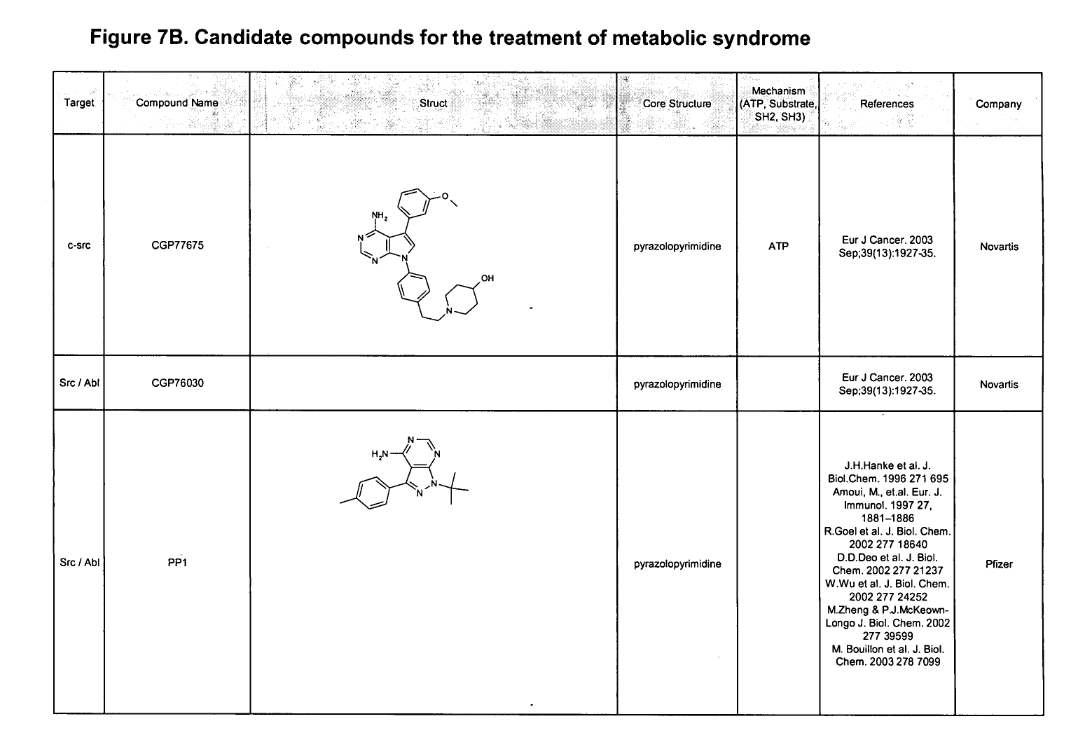

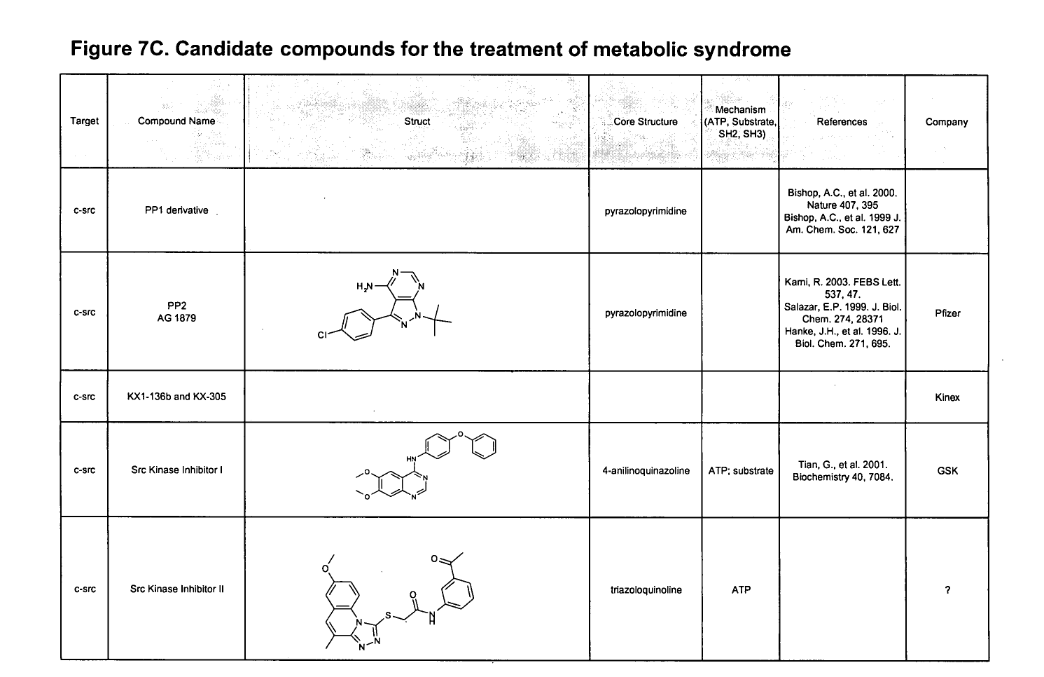

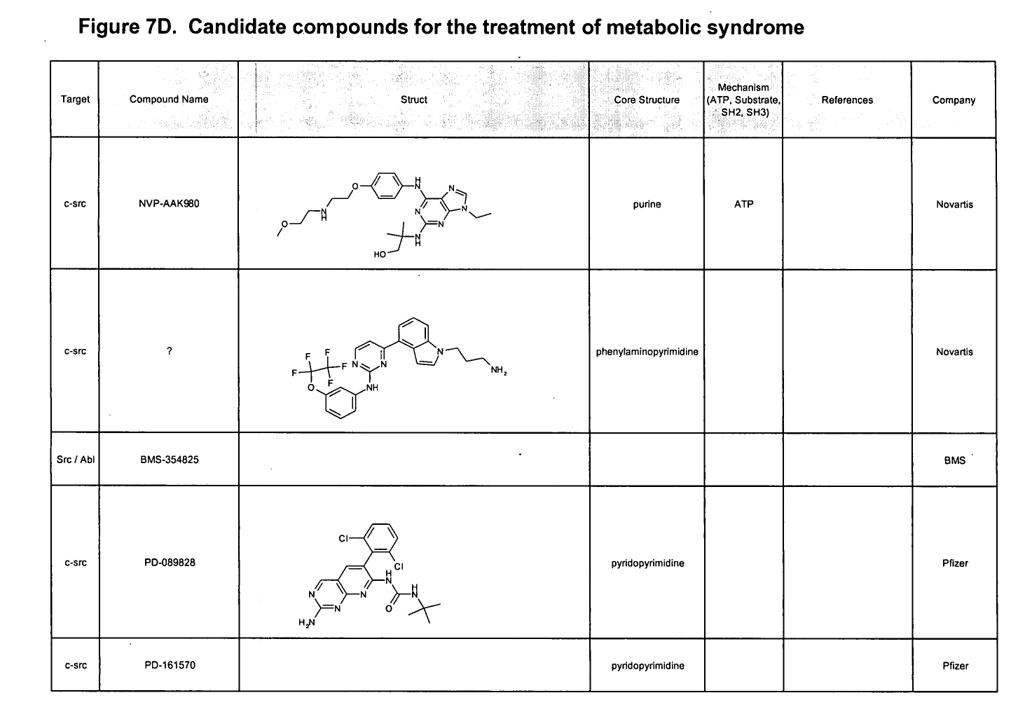

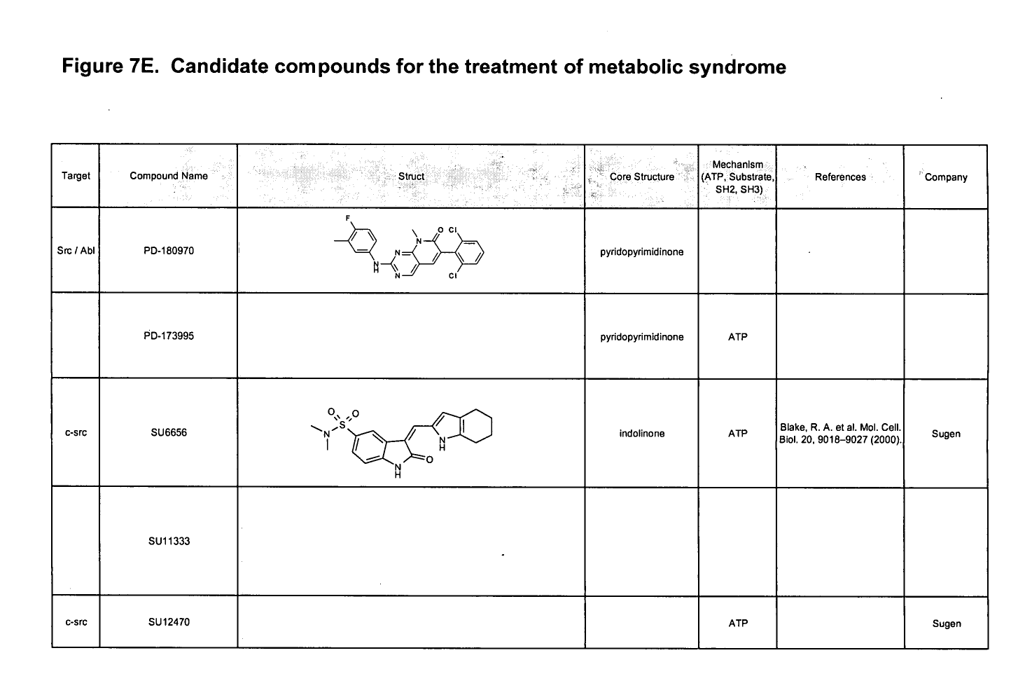

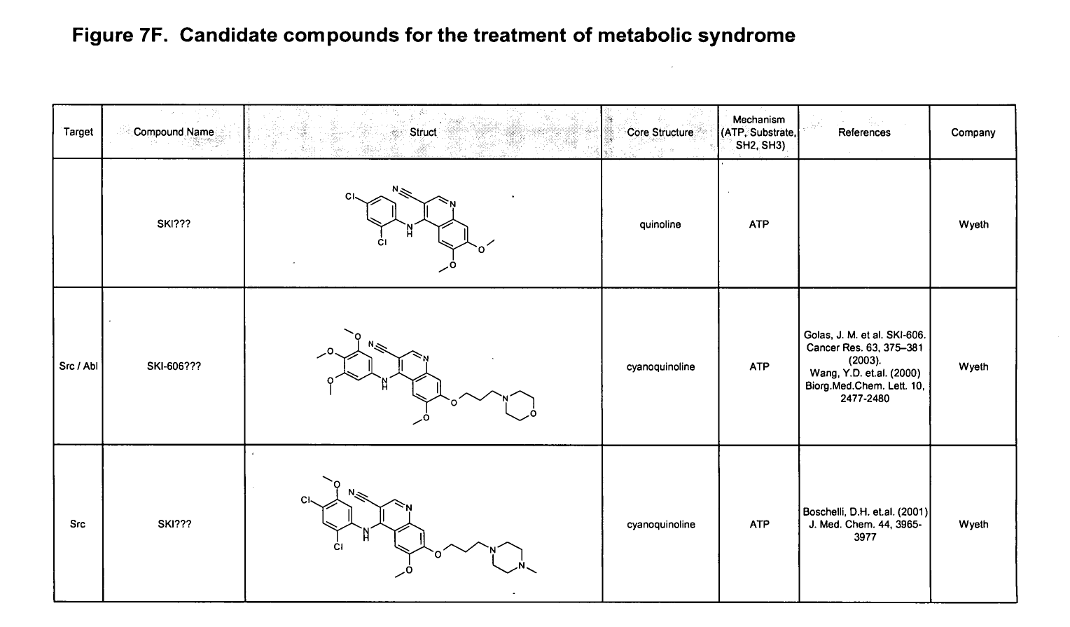

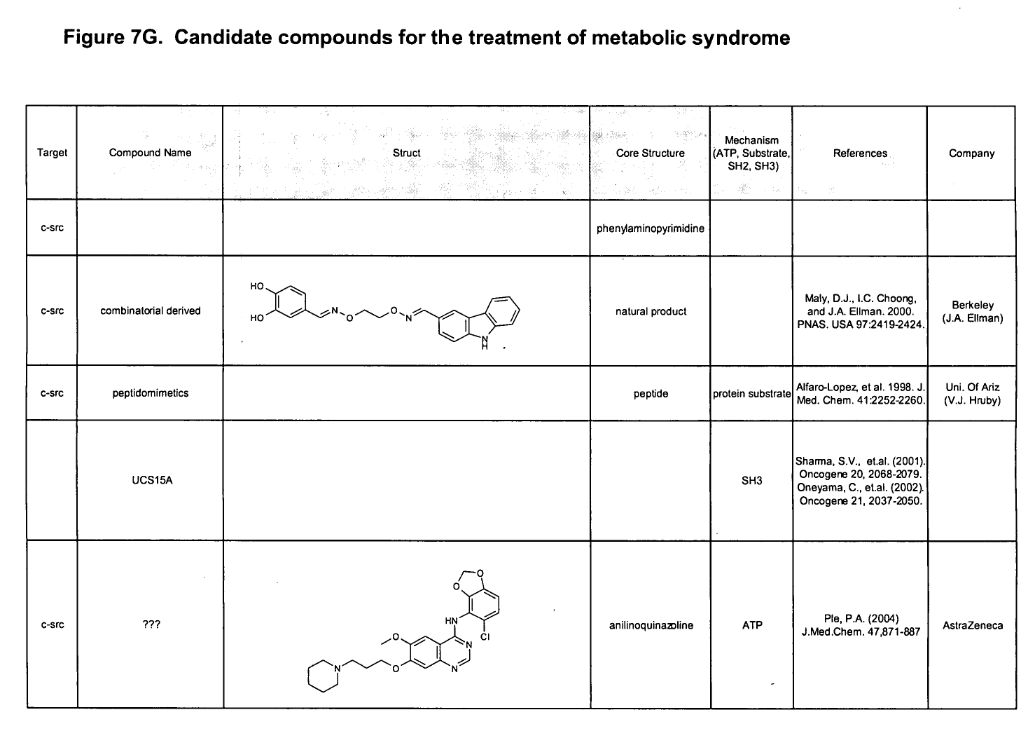

FIG. 7 (A-G) shows a number of candidate compounds; these, and derivatives and analogs thereof with suitable selectivity and adequate PK and PD properties, constitute novel drug candidates for the treatment of diabetes and obesity according to the present invention. Additional suitable compounds can be found in the References, which are incorporated herein in their entirety.

c-Src constitutes a completely novel target for drug discovery for metabolic syndrome disorders. Additional screens for selective inhibitors of c-Src and its family members can now be constructed, and the hits identified can be used in the development of new therapeutic agents for these disorders. The present invention provides for the identification of treatments for diabetes and obesity based on screening for inhibitors of c-Src. Many commercially available assays for kinase activity can be used to construct such screens; such assay techniques are well known to those skilled in the art. The hits from such screens can then be profiled against arrays of other kinases to identify selective c-Src inhibitors, and can be tested in live cell assays—such as those provided herein—to confirm their ability to activate PPAR in human cells. These compounds can then be tested in animal models of type 2 diabetes and obesity to confirm efficacy.

Protein tyrosine kinases that directly regulate the activity of the Src kinase, or which are themselves regulated by the Src kinase, constitute additional, alternative targets for the treatment of diabetes and obesity according to the present invention. For example, a kinase that phosphorylates and activates Src, or that phosphorylates another protein which in turn activates Src, constitutes an alternative target according to the invention, since inhibition of that kinase would lead to a change in the activity of Src and—through the link we identified herein—the activity of PPAR. Also any protein that is phosphorylated by Src and which lies in the pathway between Src and PPAR is an alternative target under the present invention. Therefore, alternative tyrosine kinase targets for the activation of PPARs include Pyk-2 (also known as RAFTK, CAK-b or FAK-2) which is related to the focal adhesion kinase, FAK; these two kinases are approximately 48% identical in their amino acid sequences and they have similar domain structures comprising a unique N-terminus, a centrally located catalytic domain, and two proline-rich regions at the C-terminus. Focal adhesion kinase (FAK) is a non-receptor protein tyrosine kinase discovered as a substrate for Src and as a key element of integrin signaling. FAK plays a central role in cell spreading, differentiation, migration, cell death and acceleration of the G1 to S phase transition of the cell cycle. The phosphorylation site pTyr397 is the autophosphorylation site of FAK. The site binds Src family SH2 and the p85 subunit of phosphatidyl inositol 3 kinase (PI3K). FAK is expressed in almost all tissues, whereas Pyk-2 is expressed mainly in the central nervous system and in cells and tissues of haematopoietic origin. Pyk-2 interacts with several signalling molecules and cytoskeletal proteins such as Src family protein tyrosine kinases, the adaptor proteins Grb2 and p130Cas, paxillin and the Rho-guanine nucleotide-exchange factor Graf. In response to certain stimuli, Pyk-2 also acts as an upstream activator of the mitogen-activated protein (MAP) kinase family.

Having discovered the link between c-Src and PPAR-[Gamma] it is a relatively straightforward task to determine the effect of silencing other cellular kinases on PPARs, using the methods provided herein. Other kinases found to be linked to PPAR activation can then be used in drug discovery for compounds with desired effects such as effects that mimic those of the thiazolidinediones.

Other novel chemical entities can be discovered using the present invention; in particular, by using the assays demonstrated here in a high-throughput screen of a compound library to identify additional compounds that activate PPAR-[Gamma]. Similar approaches can be taken to the identification of new pathways, targets and leads for the other members of the PPAR family, by constructing cell-based PCAs for the formation of complexes between the PPARs and their co-activators. PCA is also not the only assay alternative for the measurement of protein-protein complexes in living cells. Enzyme-fragment complementation assays can similarly be used, based on beta-galactosidase complementation technology provided by DiscoverX, Inc. (Fremont, Calif.). Other common assay techniques for this purpose include resonance energy transfer assays (FRET and BRET). If PPAR and a co-activator are fused to fluorescent proteins that undergo FRET or BRET, the induction of complex formation can be measured.

TABLE 3
Compounds for the treatment of diabetes, obesity and related conditions according to the present invention

| Patent | Author | Compound Description | Known Use |
|---|---|---|---|
| 6,660,744 | Hirst | Pyrazolopyrimidines | therapeutic agents |
| 6,713,474 | Hirst | Pyrrolopyrimidines | therapeutic agents |
| 6,706,699 | Wang | Quinolines | bone targeting src inhibitors |
| 5,710,129 | Lynch | | Inhibitors of SH2-mediated processes |
| 6,255,485 | Gray | Purines | inhibitors of protein kinases, brief mention of |
| | | | src |
| 6,638,965 | Walter | indolinones | pharmaceutical compositions |
| 6,596,746 | Das | | tyrosine kinase inhibitors |
| 6,610,724 | Salvati | | cyclin dependent kinases, and PTKs |
| 6,635,626 | Barrish | | tyrosine kinase inhibitors |
| 5,674,892 | Giese | Staursporine analogs (k252, etc) | Method and compositions for inhibiting protein |
| | | | kinases |
| | | Src Kinase Inhibitor I | |
| | | KX1-136b and KX-305 | |
| | | CGP76030 and CGP77675 | |
| 6,767,906 | Imbach | 2-amino-6-anilino-purines | |
| 6,608,071 | Altmann | Isoquinoline derivatives | angiogenesis inhibitors, one mention of “also |
| | | | inhibits src” |
| 6,686,347 | Bold | Phthalazine derivatives | VEGF mostly, SRC for treating inflammatory |
| | | | diseases |
| 5,593,997 | Dow | 4-aminopyrazolo(3-,4-D)pyrimidine | Tyrosine kinase inhibitors |
| | | and 4-aminopyrazolo-(3,4-D)pyridine | |
| 5,620,981 | Blankley | Pyrido [2,3-D]pyrimidines | inhibiting protein tyrosine kinase mediated |
| | | | cellular proliferation |
| | | PD-173995 and PD-180970 | |
| 5,326,905 | Dow | Benzylphosphonic acid | tyrosine kinase inhibitors |
| 5,719,135 | Buzzetti | 3-arylidene-7-azaoxindoles | compounds and process for their preparation |
| 6,562,818 | Bridges | Irreversible inhibitors of tyrosine | |
| | | kinases | |
| 6,683,183 | Kramer | Pyridotriazines and pyridopyridazines | Kinase inhibitors |
| 5,792,783 | Tang | 3-heteroaryl-2-indolinone SU4942, | Tyrosine Kinase Inhibitors for treatment of |
| | | SU5204, SU5416, SU4312, SU4932 | disease |
| 5,650,415 | Tang | Quinoline compounds | Tyrosine Kinase Inhibitors including src |
| 5,773,459 | Tang | Urea- and thiourea-type compounds | Tyrosine kinase inhibitors |
| 5,780,496 | Tang | quinazoline derivative | Method and compositions for inhibition of |
| | | | adaptor protein/tyrosine kinase interactions |
| 6,649,635 | McMahon | Heteroarylcarboxamide | tyrosine kinase related disorders |
| 6,656,940 | Tang | Tricyclic quinoxaline derivatives | tyrosine kinase inhibitors - ATP site |
| 6,660,763 | Tang | Bis-indolylquinone compounds | SH2 inhibit the interaction of protein tyrosine |
| | | | kinases with the GRB-2 adaptor protein |
| | | peptidomimetics | |
| 6,613,776 | Knegtel | Pyrazole compounds | kinase inhibitors |
| 6,689,778 | Bemis | see figures for structure class | Inhibitors of Src and Lck protein kinases |
| 6,638,929 | Berger | Tricyclic | kinase inhibitors |
| 6,002,008 | Wissner | Substituted 3-cyano quinolines | |
| 5,122,537 | Buzzetti | Arylvinylamide derivatives | pharmaceutical use of Tyrosine Kinase |
| | | | inhibitors |
| 5,374,652 | Buzzetti | 2-oxindole compounds | tyrosine kinase inhibitors |
| 5,397,787 | Buzzetti | Vinylene-azaindole derivatives | |
| 5,409,949 | Buzzetti | Methylen-oxindole derivatives | compositions and tyrosine kinase inhibition |
| 5,436,235 | Buzzetti | 3-aryl-glycidic ester derivatives | |
| 5,488,057 | Buzzetti | 2-oxindole compounds | tyrosine kinase activity |
| 5,627,207 | Buzzetti | Arylethenylene compounds | Tyrosine kinase inhibitors |
| 5,284,856 | Naik | 4-H-1-benzopyran-4-one derivatives | Oncogene-encoded kinases inhibition |
| 5,618,829 | Takayanagi | benzoylacrylamide derivatives | Tyrosine kinase inhibitors |
| 5,580,979 | Bachovchin | Phosphotyrosine peptidomimetics | inhibiting SH2 domain interactions |
| WO 94/03427 | | | tyrosine kinase inhibitors |
| WO 92/21660 | | | tyrosine kinase inhibitors |
| WO 91/15495 | | | tyrosine kinase inhibitors |
| WO 94/14808 | | | tyrosine kinase inhibitors |
| U.S. Pat. No. | | | tyrosine kinase inhibitors |
| 5,330,992 | | | |
| PCT WO | | bis monocyclic, bicyclic or | tyrosine kinase inhibitors. |
| 92/20642 | | heterocyclic aryl compounds | |
| PCT WO | | vinylene-azaindole derivatives | tyrosine kinase inhibitors. |
| 94/14808 | | | |
| U.S. Pat. No. | | 1-cycloproppyl-4-pyridyl-quinolones | tyrosine kinase inhibitors. |
| 5,330,992 | | | |
| U.S. Pat. No. | | Styryl compounds | tyrosine kinase inhibitors for use in the |
| 5,217,999 | | | treatment of cancer. |
| U.S. Pat. No. | | styryl-substituted pyridyl compounds | tyrosine kinase inhibitors for use in the |
| 5,302,606 | | | treatment of cancer. |
| EP Application | | quinazoline derivatives | tyrosine kinase inhibitors for use in the |
| No. 0 566 266 Al | | | treatment of cancer. |
| PCT WO | | seleoindoles and selenides | tyrosine kinase inhibitors for use in the |
| 94/03427 | | | treatment of cancer. |
| PCT WO | | tricydic polyhydroxylic compounds | tyrosine kinase inhibitors for use in the |
| 92/21660 | | | treatment of cancer. |
| PCT WO | | benzylphosphonic acid compounds | tyrosine kinase inhibitors for use in the |
| 91/15495 | | | treatment of cancer. |
| Japanese Patent | | benzylidenemalonitrile | tyrosine kinase inhibitors for use in the |
| Publication Kokai | | | treatment of cancer. |
| No. 138238/1990 | | | |
| Japanese Patent | | alpha-cyanosuccinamide derivative | tyrosine kinase inhibitors for use in the |
| Publication Kokai | | | treatment of cancer. |
| No. 222153/1988 | | | |
| Japanese Patent | | 3,5-diisopropyl-4-hydroxystyrene | tyrosine kinase inhibitors for use in the |
| Publication Kokai | | derivative | treatment of cancer. |
| No. 39522/1987 | | | |
| Japanese Patent | | 3-5-di-t-butyl-4-hydroxystyrene | tyrosine kinase inhibitors for use in the |
| Publication Kokai | | derivative | treatment of cancer. |
| No. 39523/1987 | | | |
| Japanese Patent | | Erbstatin analogue | tyrosine kinase inhibitors for use in the |
| Publication Kokai | | | treatment of cancer. |
| NO. 277347/1987 | | | |

The following patents including all those mention in the specification, published patent applications as well as all their foreign counterparts and all cited references therein are incorporated in their entirety by reference herein as if those references were denoted in the text:

US 20040161787 protein fragment complementation assays for high-throughput and high-content screening

US 20040137528 fragments of fluorescent proteins for protein fragment complementation assays

US 20040038298 protein fragment complementation assays for the detection of biological or drug interactions

US 20030108869 protein fragment complementation assay (pca) for the detection of protein-protein, protein-small molecule and protein nucleic acid interactions based on the E. coli TEM-1 beta-lactamase

US 20030049688 protein fragment complementation assays for the detection of biological or drug interactions

US 20020064769 dynamic visualization of expressed gene networks in living cells

US 20010047526 mapping molecular interactions in plants with protein fragments complementation assays

U.S. Pat. No. 6,428,951 protein fragment complementation assays for the detection of biological or drug interactions

U.S. Pat. No. 6,294,330 protein fragment complementation assays for the detection of biological or drug interactions

U.S. Pat. No. 6,270,964 protein fragment complementation assays for the detection of biological or drug interactions

What is claimed is:

1. A method of treating an individual or animal with diabetes or obesity or a metabolic syndrome, said method comprising administering to the individual or animal a therapeutically effective amount of a compound that is a protein tyrosine kinase inhibitor.

2. A method of treating diabetes or obesity or a metabolic syndrome condition in a patient by decreasing an endogenous protein tyrosine kinase activity within the patient.

3. The method of either claim 1 or 2 wherein said protein tyrosine kinase is selected from the group comprising src, abl, fps, yes, fyn, lyn, lck, blk, hck, fgr and yrk, pyk2 and FAK.

4. The method of claim 1 or claim 2, wherein said treating comprises administering at least one Src kinase inhibitor to the patient.

5. The method of claims 1-4, wherein said inhibitor is selected from the group consisting of PP2, KX1-136b, KX-305, CGP76030, CGP77675, NVP-AAK980, PD-089828, PD-161570, PD-173995, PD-180970, SU6656, SKI-606 and a derivative, analog or metabolite thereof.

6. The method of claim 3, wherein the inhibitor is selected from the group consisting of an antisense oligonucleotide, a small interfering RNA molecule, a chemical compound, a polypeptide, and a function-blocking antibody or fragment thereof.

7. The method of claim 3, wherein said treating comprises administering a polynucleotide encoding at least one Src inhibitor to the patient, wherein the polynucleotide is expressed within the patient.

8. The method of claims 1-3, wherein the patient is human.

9. A method of screening for compounds useful in the treatment of human disease, said method comprising (a) constructing an assay to measure activation of a nuclear hormone receptor; (b) contacting a cell with an siRNA targeting a cellular gene of interest; (c) detecting the effect of said siRNA in said assay; (d) determining that said cellular gene of interest is an potential drug target, if said siRNA produces an effect on said nuclear hormone receptor, wherein said effect is substantially similar to the effect of a known ligand of said nuclear hormone receptor.

10. A method of screening for compounds useful in the treatment of diabetes and obesity, said method comprising (a) constructing an assay for c-Src or a family member of c-Src; (b) contacting said assay with one or more chemical compound(s); (c) identifying a compound that inhibits c-Src or a family member of c-Src.

11. A method according to claim 9 wherein said assay is a measurement of a protein-protein interaction or a protein-protein complex.

12. An assay for identifying a compound that modulates the activity of a peroxisome-proliferator activated receptor (PPAR), said assay comprising (a) contacting a cell or a cell lysate containing a PPAR polypeptide and a PPAR-associated polypeptide with a test agent; and (b) detecting one or more of the following characteristics (i) the level of said PPAR polypeptide; (ii) the amount of the complex between said PPAR polypeptide and said PPAR-associated polypeptide; (iii) in the case of the cell, the subcellular location of said PPAR polypeptide; (iv) in the case of the cell, the subcellular location of the complex between said PPAR polypeptide and said PPAR-associated polypeptide; (v) the level of DNA binding activity of said PPAR; wherein a change in one or more of said characteristics in the presence of the test agent, relative to the absence of the test agent, indicates that the test agent is a compound that modulates the activity of a PPAR.

13. A method for inhibiting expression, in a eukaryotic cell, of a gene whose transcription is regulated by a PPAR, the method comprising reducing the activity of a protein tyrosine kinase in said cell such that expression of said gene is inhibited.

14. The method according to claim 13 wherein said protein tyrosine kinase is a Src kinase or a member of the Src kinase family or a Pyk2 kinase or a member of the FAK family of kinases.
